(19) United States
(12) Patent Application Publication
(10) Pub. No.: US 2009/0004300 A1
(43) Pub. Date: Jan. 1, 2009

(54) Method and Composition for Enhancing Fertility

(76) Inventor: Ai Ja Lee, Cliffside Park, NJ (US)

Correspondence Address: David Chen;Delio and Peterson LLC, 121 Whitney Avenue, New Haven, CT, 06510-1214, US

(21) Appl. No.: 12/173,820

(22) Filed: Jul. 16, 2008

Related U.S. Application Data
(63) Continuation-in-part of application No. 10/955,871, filed on Sep. 30, 2004, now Pat. No. 7,410,657.

(51) Int. Cl. A61K 36/258

(52) U.S. Cl. 424/728

(57) ABSTRACT

The invention relates to a method and composition for enhancing fertility, including the steps of preparing a formula by mixing between approximately 2 grams and approximately 10 grams of each of the following components: Radix Ginseng Rubra, Semen Ziziphi Spinosae, Radix Angelicae Sinensis, Radix Astragali, Rhizoma Atractylodis, Screrotium Poriae Cocos, Radix Glycyrrhizae Uralensis, Semen Persicae, Flos Carthami Tinctorii, Folium Artemisiae, Cornu Cervi Parvum, and combinations thereof; and preparing a second formula by mixing between approximately 2 grams and approximately 10 grams of each of the following components: Radix Ginseng Rubra, Rhizoma Atractylodis, Folium Perillae, Radix Scutellariae Baicalensis, Radix Glycyrrhizae Uralensis, Semen Cuscutae, Radix Astragali, Radix Dipsaci, and combinations thereof.

### CROSS-REFERENCE TO RELATED APPLICATIONS

This application is a continuation in part of co-pending U.S. patent application Ser. No. 10/955,871 entitled “Alternative Remedy-Based composition for Enhancing Fertility” filed Sep. 30, 2004, the content of which is incorporated herein by reference.

### FIELD OF THE INVENTION

The invention relates to a method and composition of herbs for enhancing fertility.

### BACKGROUND

It is estimated that 1 in 6 couples experience difficulties in conceiving a child. Some of the difficulties may be linked to infertility in a woman caused by a medical condition, for example. Other difficulties, however, are not related to infertility and may be attributed to everyday stress, unhealthy diets, use of tobacco or alcohol, etc. In this case, what seems like interminable delay in getting pregnant may cause additional stress and frustration, exacerbating the situation and spiraling further into a proverbial vicious circle.

While prescription or over-the-counter drugs may be efficacious in addressing the fertility problems, alternative medicine based on herbs and other natural ingredients appears to be more preferable from the overall health viewpoint. Moreover, some patients who previously experienced difficulty in conceiving or maintaining a successful pregnancy while being treated with more mainstream or Western medicine often conceive or bring the baby to term when treated with herbal or alternative remedies. In some cases, herbal compositions are now part of a health regimen by two out of five Americans and have become more acceptable to patients who traditionally shied away from alternative treatments.

A need therefore exists for an alternative medicine-based, natural solution to the above difficulties experienced by a woman trying to conceive a child.

### SUMMARY OF THE INVENTION

It is therefore an object of the invention to provide an herbal remedy to enhance fertility.

It is another object to provide an herbal remedy that facilitates conception and bringing a fetus/baby to full term.

Yet another object is a composition and method for administering the composition to enhance fertility.

These and other objects of the invention are achieved by a method for enhancing fertility, including the step of preparing a formula by mixing between approximately 2 grams and approximately 10 grams of each of the following components: Radix Ginseng Rubra, Semen Ziziphi Spinosae, Radix Angelicae Sinensis, Radix Astragali, Rhizoma Atractylodis, Screrotium Poriae Cocos, Radix Glycyrrhizae Uralensis, Semen Persicae, Flos Carthami Tinctorii, Folium Artemisiae, Cornu Cervi Parvum, and combinations thereof.

In some embodiments, the method includes the step of administering the formula; preparing a second formula by mixing between approximately 2 grams and approximately 10 grams of each of the following components: Radix Ginseng Rubra, Rhizoma Atractylodis, Folium Perillae, Radix Scutellariae Baicalensis, Radix Glycyrrhizae Uralensis, Semen Cuscutae, Radix Astragali, Radix Dipsaci, and combinations thereof; and administering the second formula following administering the formula.

In an optional embodiment, the method reduces the formula to a liquid state by heating the mixture; and reduces the second formula to a liquid state by heating the mixture.

In a further embodiment, the method also adds to the formula between approximately 2 grams and approximately 10 grams of each of the following: Radix Rehmanniae Glutinosae Conquitae, Fructus Lycii Chinensis, Semen Cuscutae, and combinations thereof.

In another embodiment, the method also adds to the formula between approximately 2 grams and approximately 10 grams of each of the following: Radix Rehmanniae Glutinosae Conquitae, Fructus Lycii Chinensis, Semen Cuscutae, Radix Polygalae Tenuifoliae, Rhizoma Sparganii, Rhizoma Curcumae Zedoariae, Radix Ligustici Wallichii, Radix Paeoniae Rubra, Fructus Ligusti Lucidi, and combinations thereof.

In yet another embodiment, the method adds to the second formula between approximately 2 grams and approximately 10 grams of each of the following: Ramus Loranti Seu Visci, Cornu Cervi Parvum, Cortex Eucommiae Ulmoidis, Herba Cistanches, Fructus Amomi, and combinations thereof.

In an optional embodiment, the method adds to the second formula between approximately 2 grams and approximately 10 grams of each of the following: Ramus Loranti Seu Visci, Cornu Cervi Parvum, Cortex Eucommiae Ulmoidis, Pericarpium Citri Reticulatae, Fructus Schisandrae Chinensis, Herba Cistanches, Fructus Amomi, Rhizoma Pinelliae Ternatae, Fructus Lycii Chinensis, and combinations thereof.

In a more specific embodiment, the method includes the steps of mixing between approximately 6 grams and approximately 10 grams of each of the following components: Radix Ginseng Rubra, Semen Ziziphi Spinosae, Radix Angelicae Sinensis, Radix Astragali, Rhizoma Atractylodis, Screrotium Poriae Cocos, Radix Glycyrrhizae Uralensis, Semen Persicae, Flos Carthami Tinctorii, Folium Artemisiae, Cornu Cervi Parvum, and combinations thereof; and mixing between approximately 6 grams and approximately 10 grams of each of the following components: Radix Ginseng Rubra, Rhizoma Atractylodis, Folium Perillae, Radix Scutellariae Baicalensis, Radix Glycyrrhizae Uralensis, Semen Cuscutae, Radix Astragali, Radix Dipsaci, and combinations thereof.

In another aspect of the invention, a composition for enhancing fertility has a formula of between approximately 2 grams and approximately 10 grams of each of the following: Radix Ginseng Rubra, Semen Ziziphi Spinosae, Radix Angelicae Sinensis, Radix Astragali, Rhizoma Atractylodis, Screrotium Poriae Cocos, Radix Glycyrrhizae Uralensis, Semen Persicae, Flos Carthami Tinctorii, Folium Artemisiae, Cornu Cervi Parvum, and combinations thereof.

In some embodiments, the composition further has between approximately 2 grams and approximately 10 grams of each of the following: Radix Rehmanniae Glutinosae Conquitae, Fructus Lycii Chinensis, Semen Cuscutae, and combinations thereof.

In another embodiment, the composition has between approximately 2 grams and approximately 10 grams of each of the following: Radix Rehmanniae Glutinosae Conquitae, Fructus Lycii Chinensis, Semen Cuscutae, Radix Polygalae Tenuifoliae, Rhizoma Sparganii, Rhizoma Curcumae Zedoariae, Radix Ligustici Wallichii, Radix Paeoniae Rubra, Fructus Ligusti Lucidi, and combinations thereof.

In a more specific embodiment, the composition has between approximately 6 grams and approximately 10 grams of each of the following: Radix Ginseng Rubra, Semen Ziziphi Spinosae, Radix Angelicae Sinensis, Radix Astragali, Rhizoma Atractylodis, Screrotium Poriae Cocos, Radix Glycyrrhizae Uralensis, Semen Persicae, Flos Carthami Tinctorii, Folium Artemisiae, Cornu Cervi Parvum, and combinations thereof.

In another aspect of the invention, the composition for enhancing fertility includes a formula of between approximately 2 grams and approximately 10 grams of each of the following: Radix Ginseng Rubra, Rhizoma Atractylodis, Folium Perillae, Radix Scutellariae Baicalensis, Radix Glycyrrhizae Uralensis, Semen Cuscutae, Radix Astragali, Radix Dipsaci, and combinations thereof.

In some embodiments, the composition includes between approximately 2 grams and approximately 10 grams of each of the following: Ramus Loranti Seu Visci, Cornu Cervi Parvum, Cortex Eucommiae Ulmoidis, Herba Cistanches, Fructus Amomi, and combinations thereof.

In further embodiments, the compositions has between approximately 2 grams and approximately 10 grams of each of the following: Ramus Loranti Seu Visci, Cornu Cervi Parvum, Cortex Eucommiae Ulmoidis, Pericarpium Citri Reticulatae, Fructus Schisandrae Chinensis, Herba Cistanches, Fructus Amomi, Rhizoma Pinelliae Ternatae, Fructus Lycii Chinensis, and combinations thereof.

In a narrower embodiment, the composition has between approximately 6 and approximately 10 grams of each of the following components: Radix Ginseng Rubra, Rhizoma Atractylodis, Folium Perillae, Radix Scutellariae Baicalensis, Radix Glycyrrhizae Uralensis, Semen Cuscutae, Radix Astragali, Radix Dipsaci, and combinations thereof.

### DETAILED DESCRIPTION

In accordance with one embodiment of the present invention, a number of herbs are combined to produce a composition that enhances and promotes fertility in females. According to this embodiment, one herbal composition comprises 40 herbs, the so-called “A” formula.

In another embodiment, the composition inhibits miscarriage once conception has been achieved, wherein the composition comprises another set of 40 herbs, the so-called “B” formula. Some herbs are present in both formulas. In a further embodiment, the composition both promotes fertility and inhibits miscarriage, in which case the herbal composition comprises a first part, including formula A, and a second part, including formula B.

“A” formula is typically administered daily for 2 months for conception to occur. After conception, “B” formula is administered daily for 1 month to prevent miscarriage. Preferably, the B formula is administered immediately after discovering the female is pregnant. If, however, conception does not occur after 2 months, “A” formula is continued in some embodiments for one more month.

Each herbal ingredient in the “A” and “B” formulas is approximately between 3 and 10 grams in dry weight. To be more effective, the exact weight for each herbal ingredient depends on the female's weight. For an average female, each herbal ingredient is approximately 4 grams in weight.

According to one embodiment of the present invention, each “A” and “B” formula weighs approximately 164 grams, comprising the below-mentioned herbal ingredients. Such “raw” herbal combination in each formula is boiled in hot water to produce approximately 18 ounces of liquid, which represents a daily dosage. In some of these embodiments, the 18 ounces is administered 3 times a daily, 6 ounces at a time, to facilitate ingestion.

Both formulas are administered orally in the preferred embodiment of the present invention. According to additional embodiments of the present invention, each formula may be administered in capsules, pills, caplets, tablets, and the like forms, as known to those skilled in the art.

The “A” formula comprises the following herbs listed in no particular order according to their pharmaceutical names:

### 1) Radix Polygalae Tenuifoliae

### 2) Semen Ziziphi Spinosae

### 3) Radix Ginseng

### 4) Rhizoma Atractylodis Macrocephalae

### 5) Rhizoma Zingiberis Officinalis Recens

### 6) Screrotium Poriae Cocos

### 7) Radix Astragali

### 8) Radix Ligustici Wallichii

### 9) Radix Angelicae Sinensis

### 10) Radix Rehmanniae Glutino sac Conquitae

### 11) Radix Paeoniae Rubra

### 12) Radix Bupleuri

### 13) Ramulus Cinnamomi Cassiae

### 14) Arillus Euphoriae Longanae

### 15) Radix Saussureae Seu Vladimiriae

### 16) Fructus Lycii Chinensis

### 17) Fructus Schisandrae Chinensis

### 18) FructusRubi

### 19) Semen Cnidii Monnieri

### 20) Semen Cuscutae

### 21) Semen Persicae

### 22) Flos Carthami Tinctorii

### 23) Folium Artemisiae

### 24) Herba Agastaches Seu Pogostemi

### 25) Folium Periulae Frutescentis

### 26) Herba Leonuri Heterophylli

### 27) Rhizoma Cyperi Rotundi

### 28) Radix Glycyrrhizae Uralensis

### 28) Fructus Ziziphi Jujubae

### 30) Fructus Foeniculi Vulgaris

### 31) Fructus Evodiae Rutaecarpae

### 32) Semen Plantaginis

### 33) Radix Ledebouriellae Sesloidis

### 34) Radix Scutellariae Baicalensis

### 35) Tuber Asparagi Cochinchinensis

### 36) Massa Fermentata

### 37) Fructus Hordei Vulgaris Genninantus

### 38) Rhizoma Sparganii

### 39) Rhizoma Curcumae Zedoariae

### 40) Cornu Cervi Parvum

The “B” formula comprises the following herbs listed in no particular order according to their pharmaceutical names:

### 1) Radix Polygalae Tenuifoliae

### 2) Semen Ziziphi Spinosae

### 3) Radix Ginseng

### 4) Rhizoma Atractylodis Macrocephalae

### 5) Rhizoma Zingiberis Officinalis Recens

### 6) Screrotium Poriae Cocos

### 7) Radix Astragali

### 8) Radix Ligustici Wallichii

### 9) Radix Angelicae Sinensis

### 10) Radix Rehmanniae Glutinosae Conquitae

### 11) Radix Paeoniae Lactiflorae

### 12) Radix Bupleuri

### 13) Cortex Moutan Radicis

### 14) Arillus Euphoriae Longanae

### 15) Radix Saussureae Seu Vladimiriae

### 16) Fructus Lycii Chinensis

### 17) Fructus Schisandrae Chinensis

### 18) FructusRubi

### 19) Semen Cnidii Monnieri

### 20) Semen Cuscutae

### 21) Cortex Eucommiae Ulmoidis

### 22) Gelatinum Asini

### 23) Folium Artemisiae

### 24) Herba Leonuri Heterophylli

### 25) Folium Perillae Frutescentis

### 26) Herba Agastaches Seu Pogostemi

### 27) Rhizoma Cypen Rotundi

### 28) Fructus Foeniculi Vulgaris

### 29) Fructus Evodiae Rutaecarpae

### 30) Radix Glycyrrhizae Uralensis

### 31) Fructus Ziziphi Jujubae

### 32) Semen Plantaginis

### 33) Os Draconis

### 34) Concha Ostreae

### 35) Radix Scutellariae Baicalensis

### 36) Pericarpium Citri Reticulatae

### 37) Ramus Loranthu Seu Visci

### 38) Cornu Cervi Parvum

### 39) Rhizoma Atractylodis

### 40) Fructus Seu Semen Amomi

In another embodiment, subsets or smaller numbers of herbs from formulas A and/or B are utilized. In some of these embodiments, this is advantageous when a patient is allergic to some of the above listed 40 herbs but a smaller amount, or subset, of herbs still provides an effective amount for achieving pregnancy and/or inhibiting miscarriage.

In other embodiments, a smaller amount of herbs is needed to be ingested for other reasons, such as undesired reactions with foods. The above listed herbs for formula A is taken daily. Likewise, the above listed herbs for formula B are taken daily.

In a further embodiment, new herbs are added to the subsets or smaller numbers of herbs from formulas A and/or B to further enhance body absorption, ingestion, or inhibit undesired reactions with a patient.

In one aspect, formula A comprises 20 herbs, which is a subset of the above listed 40 herbs plus additional new herbs. Namely, Radix Ginseng Rubra, Semen Ziziphi Spinosae, Radix Angelicae Sinensis, Radix Astragali, Rhizoma Atractylodis, Screrotium Poriae Cocos, Radix Glycyrrhizae Uralensis, Semen Persicae, Flos Carthami Tinctorii, Folium Artemisiae, Cornu Cervi Parvum, Radix Rehmanniae Glutinosae Conquitae, Fructus Lycii Chinensis, Semen Cuscutae, Radix Polygalae Tenuifoliae, Rhizoma Sparganii, Rhizoma Curcumae Zedoariae, Radix Ligustici Wallichii, Radix Paeoniae Rubra, Fructus Ligusti Lucidi, and combinations thereof. Optionally, this aspect includes all 20 of these listed herbs. In another aspect, formula A comprises 14 herbs, which is a subset of the above listed 40 herbs plus additional new herbs. Namely, Radix Ginseng Rubra, Semen Ziziphi Spinosae, Radix Angelicae Sinensis, Radix Astragali, Rhizoma Atractylodis, Screrotium Poriae Cocos, Radix Glycyrrhizae Uralensis, Semen Persicae, Flos Carthami Tinctorii, Folium Artemisiae, Cornu Cervi Parvum, Radix Rehmanniae Glutinosae Conquitae, Fructus Lycii Chinensis, Semen Cuscutae, and combinations thereof. Optionally, this aspect includes all 14 of these listed herbs.

In a further aspect, formula A comprises 11 herbs, which is a subset of the above listed 40 herbs plus additional new herbs. Namely, Radix Ginseng Rubra, Semen Ziziphi Spinosae, Radix Angelicae Sinensis, Radix Astragali, Rhizoma Atractylodis, Screrotium Poriae Cocos, Radix Glycyrrhizae Uralensis, Semen Persicae, Flos Carthami Tinctorii, Folium Artemisiae, Cornu Cervi Parvum, and combinations thereof. Optionally, this aspect includes all 11 of these listed herbs.

In one aspect, formula B comprises 17 herbs, which is a subset of the above listed 40 herbs plus additional new herbs. Namely, Radix Ginseng Rubra, Rhizoma Atractylodis, Folium Perillae, Radix Scutellariae Baicalensis, Ramus Loranti Seu Visci, Radix Glycyrrhizae Uralensis, Semen Cuscutae, Radix Astragali, Cornu Cervi Parvum, Cortex Eucommiae Ulmoidis, Pericarpium Citri Reticulatae, Fructus Schisandrae Chinensis, Herba Cistanches, Radix Dipsaci, Fructus Amomi, Rhizoma Pinelliae Ternatae, Fructus Lycii Chinensis, and combinations thereof. Optionally, this aspect includes all 17 of these listed herbs.

In another aspect, formula B comprises 13 herbs, which is a subset of the above listed 40 herbs plus additional new herbs. Namely, Radix Ginseng Rubra, Rhizoma Atractylodis, Folium Perillae, Radix Scutellariae Baicalensis, Ramus Loranti Seu Visci, Radix Glycyrrhizae Uralensis, Semen Cuscutae, Radix Astragali, Cornu Cervi Parvum, Cortex Eucommiae Ulmoidis, Herba Cistanches, Radix Dipsaci, Fructus Amomi, and combinations thereof. Optionally, this aspect includes all 13 of these listed herbs.

In a further aspect, formula B comprises 8 herbs, which is a subset of the above listed 40 herbs plus additional new herbs. Namely, Radix Ginseng Rubra, Rhizoma Atractylodis, Folium Perillae, Radix Scutellariae Baicalensis, Radix Glycyrrhizae Uralensis, Semen Cuscutae, Radix Astragali, Radix Dipsaci, and combinations thereof. Optionally, this aspect includes all 8 of these listed herbs.

Although Radix Ginseng is listed as one of the full set of 40 herbs of formulas A and B, it also is used in the above subsets in a variation of formula A. Radix Ginseng Rubra is a red color of the Radix Ginseng herb wherein Radix Ginseng is white and wherein the red Ginseng is found, in some embodiments, to enhance ingestion and reduce irritation.

In each of the above aspects concerning formula A and/or formula B, a preferred amount of each herb is between approximately 2 grams and approximately 10 grams. A more preferred amount of each herb is between approximately 4 grams and approximately 9 grams. An even more preferred amount of each herb is between approximately 6 grams and approximately 8 grams. A most preferred amount of each herb is approximately 8 grams.

In one embodiment, an average female is found to weigh between approximately 100 and approximately 150 pounds (1 pound is about 16 ounces) and is administered a daily amount of approximately 8 grams of each herb of formula A or B. In a second embodiment, an above average female is found to weigh above approximately 150 pounds and is administered a daily amount of approximately 10 grams of each herb of formula A or B. In a third embodiment, a below average female is found to weigh less than approximately 100 pounds and is administered a daily amount of approximately 6 grams of each herb of formula A or B.

In some embodiments, all of the herbs are ground into powder form from plants. Once in powder form, the herbs are placed in a capsule, pill, caplet, tablet, or other vehicle for ingestion. In other embodiments the herbs are mixed with a liquid and dissolved.

Regardless of the manner for ingestion, whether in powder or liquid form, formula A or formula B is taken daily. Moreover, although formula A or B may be divided into 2 or more parts or quantities to facilitate digestion, the total daily consumption should equal the above described daily amount for formula A or B.

While the invention has been described and illustrated in connection with the above embodiments, many variations and modifications may be made without departing from the spirit and scope of the invention, and the invention is thus not to be limited to the precise details of methodology or construction set forth above as such variations and modification are intended to be included within the scope of the invention.

What is claimed is:

1. A method for enhancing fertility, comprising the step of:
preparing a formula by mixing between approximately 2 grams and approximately 10 grams of each of the following components: Radix Ginseng Rubra, Semen Ziziphi Spinosae, Radix Angelicae Sinensis, Radix Astragali, Rhizoma Atractylodis, Screrotium Poriae Cocos, Radix Glycyrrhizae Uralensis, Semen Persicae, Flos Carthami Tinctorii, Folium Artemisiae, Cornu Cervi Parvum, and combinations thereof.

2. The method of claim 1, further comprising the step of:
administering the formula;
preparing a second formula by mixing between approximately 2 grams and approximately 10 grams of each of the following components: Radix Ginseng Rubra, Rhizoma Atractylodis, Folium Perillae, Radix Scutellariae Baicalensis, Radix Glycyrrhizae Uralensis, Semen Cuscutae, Radix Astragali, Radix Dipsaci, and combinations thereof; and
administering the second formula following administering the formula.

3. The method according to claim 2, further comprising the steps of reducing the formula to a liquid state by heating the mixture; and reducing the second formula to a liquid state by heating the mixture.

4. The method according to claim 1, further comprising the step of adding to the formula between approximately 2 grams and approximately 10 grams of each of the following: Radix Rehmanniae Glutinosae Conquitae, Fructus Lycii Chinensis, Semen Cuscutae, and combinations thereof.

5. The method according to claim 1, further comprising the step of adding to the formula between approximately 2 grams and approximately 10 grams of each of the following: Radix Rehmanniae Glutinosae Conquitae, Fructus Lycii Chinensis, Semen Cuscutae, Radix Polygalae Tenuifoliae, Rhizoma Sparganii, Rhizoma Curcumae Zedoariae, Radix Ligustici Wallichii, Radix Paeoniae Rubra, Fructus Ligusti Lucidi, and combinations thereof.

6. The method according to claim 2, further comprising the step of adding to the second formula between approximately 2 grams and approximately 10 grams of each of the following: Ramus Loranti Seu Visci, Cornu Cervi Parvum, Cortex Eucommiae Ulmoidis, Herba Cistanches, Fructus Amomi, and combinations thereof.

7. The method according to claim 2, further comprising the step of adding to the second formula between approximately 2 grams and approximately 10 grams of each of the following: Ramus Loranti Seu Visci, Cornu Cervi Parvum, Cortex Eucommiae Ulmoidis, Pericarpium Citri Reticulatae, Fructus Schisandrae Chinensis, Herba Cistanches, Fructus Amomi, Rhizoma Pinelliae Ternatae, Fructus Lycii Chinensis, and combinations thereof.

8. The method according to claim 2, further comprising the steps of mixing between approximately 6 grams and approximately 10 grams of each of the following components: Radix Ginseng Rubra, Semen Ziziphi Spinosae, Radix Angelicae Sinensis, Radix Astragali, Rhizoma Atractylodis, Screrotium Poriae Cocos, Radix Glycyrrhizae Uralensis, Semen Persicae, Flos Carthami Tinctorii, Folium Artemisiae, Cornu Cervi Parvum, and combinations thereof; and
mixing between approximately 6 grams and approximately 10 grams of each of the following components: Radix Ginseng Rubra, Rhizoma Atractylodis, Folium Perillae, Radix Scutellariae Baicalensis, Radix Glycyrrhizae Uralensis, Semen Cuscutae, Radix Astragali, Radix Dipsaci, and combinations thereof.

9. A composition for enhancing fertility, comprising:
a formula of between approximately 2 grams and approximately 10 grams of each of the following: Radix Ginseng Rubra, Semen Ziziphi Spinosae, Radix Angelicae Sinensis, Radix Astragali, Rhizoma Atractylodis, Screrotium Poriae Cocos, Radix Glycyrrhizae Uralensis, Semen Persicae, Flos Carthami Tinctorii, Folium Artemisiae, Cornu Cervi Parvum, and combinations thereof.

10. The composition according to claim 9, further comprising between approximately 2 grams and approximately 10 grams of each of the following: Radix Rehmanniae Glutinosae Conquitae, Fructus Lycii Chinensis, Semen Cuscutae, and combinations thereof.

11. The composition according to claim 9, further comprising between approximately 2 grams and approximately 10 grams of each of the following: Radix Rehmanniae Glutinosae Conquitae, Fructus Lycii Chinensis, Semen Cuscutae, Radix Polygalae Tenuifoliae, Rhizoma Sparganii, Rhizoma Curcumae Zedoariae, Radix Ligustici Wallichii, Radix Paeoniae Rubra, Fructus Ligusti Lucidi, and combinations thereof.

12. The composition according to claim 9, further comprising between approximately 6 grams and approximately 10 grams of each of the following: Radix Ginseng Rubra, Semen Ziziphi Spinosae, Radix Angelicae Sinensis, Radix Astragali, Rhizoma Atractylodis, Screrotium Poriae Cocos, Radix Glycyrrhizae Uralensis, Semen Persicae, Flos Carthami Tinctorii, Folium Artemisiae, Cornu Cervi Parvum, and combinations thereof.

13. A composition for enhancing fertility, comprising:
a formula of between approximately 2 grams and approximately 10 grams of each of the following: Radix Ginseng Rubra, Rhizoma Atractylodis, Folium Perillae, Radix Scutellariae Baicalensis, Radix Glycyrrhizae Uralensis, Semen Cuscutae, Radix Astragali, Radix Dipsaci, and combinations thereof.

14. The composition according to claim 13, further comprising between approximately 2 grams and approximately 10 grams of each of the following: Ramus Loranti Seu Visci, Cornu Cervi Parvum, Cortex Eucommiae Ulmoidis, Herba Cistanches, Fructus Amomi, and combinations thereof.

15. The composition according to claim 13, further comprising between approximately 2 grams and approximately 10 grams of each of the following: Ramus Loranti Seu Visci, Cornu Cervi Parvum, Cortex Eucommiae Ulmoidis, Pericarpium Citri Reticulatae, Fructus Schisandrae Chinensis, Herba Cistanches, Fructus Amomi, Rhizoma Pinelliae Ternatae, Fructus Lycii Chinensis, and combinations thereof.

16. The composition according to claim 13, further comprising between approximately 6 and approximately 10 grams of each of the following components: Radix Ginseng Rubra, Rhizoma Atractylodis, Folium Perillae, Radix Scutellariae Baicalensis, Radix Glycyrrhizae Uralensis, Semen Cuscutae, Radix Astragali, Radix Dipsaci, and combinations thereof.
